(19) United States
(12) Patent Application Publication
(10) Pub. No.: US 2023/0148964 A1
(43) Pub. Date: May 18, 2023

(54) AUDIBLE AND VISUAL CUES IN CHEST-WORN SENSOR DEVICE

(71) Applicant: Analog Devices, Inc., Wilmington, MA (US)

(72) Inventor: Deborah L. PENNY, Winchester, MA (US)

(73) Assignee: Analog Devices, Inc., Wilmington, MA (US)

(21) Appl. No.: 17/527,901

(22) Filed: Nov. 16, 2021

(51) Int. Cl. A61B 5/00; G08B 7/06; G08B 21/02; A61B 7/04

(52) U.S. Cl. CPC A61B 5/684; G08B 7/06; G08B 21/02; A61B 5/6843; A61B 5/6823; A61B 5/4884; A61B 7/04

(57) ABSTRACT

A chest-worn sensor device, such as a device for daily at-home monitoring of cardiopulmonary health conditions, includes an audio and/or visual interface that provides cues to assist a user in performing a measurement sequence. The sensor device may detect whether there are any body posture issues or device contact issues. In response to detecting an issue, the sensor device provides and audio and/or visual cue to the user indicating the issue. The sensor device may provide additional cues throughout the measurement sequence, such as a cue that a measurement is being taken, a cue for the user to change body posture, and a cue for the user to remove the sensor device.

### FIELD OF THE DISCLOSURE

The present application relates generally to user interfaces including audio and visual cues provided by a chest-worn sensor for managing medical or health conditions in human subjects.

### BACKGROUND

Congestive heart failure (CHF) is a known cardiac condition in which damaged heart muscle loses its ability to pump sufficient amounts of blood to meet the body's demands. In the early stages of CHF, such an inability to pump sufficient amounts of blood may occur only while a human exercises. However, in more advanced stages of CHF, an inability to pump sufficient amounts of blood may occur even while the human subject is at rest. CHF is one of the most commonly diagnosed cardiac conditions in hospital patients over the age of 65, and one of the most frequent reasons for such patients' readmission to hospitals in a time duration of 30 days. In recent years, 30-day hospital readmission expenses for CHF have increased to $1.8 billion per year, with approximately $13,000 being allotted for each readmission at a 25% readmission rate. Some of the reasons for such patients' readmission to hospitals can include, but are not limited to, (1) patient non-compliance with regard to diet and medication, which can result in excess fluid in the lungs or extreme dehydration, (2) incomplete titration of medication dosages, which often need to be modified as a patient moves from the hospital environment back to his or her home, and (3) atrial fibrillation, which can onset after the patient's discharge from the hospital.

Management of CHF in patients following discharge from the hospital has traditionally focused on monitoring the patients' fluid retention using sensors incorporated in implantable cardiac devices, such as implantable cardioverter defibrillators (ICDs), cardiac resynchronization therapy-defibrillators (CRT-Ds), or pacemakers. Such implantable cardiac devices can detect developing pulmonary congestion in a patient by measuring the patient's thoracic fluid impedance. For example, an implantable cardiac device such as an ICD, CRT-D, or pacemaker can be configured to pass an electrical current across a patient's lung, and to measure the resulting intra-thoracic impedance. As the patient's thoracic fluid accumulates during pulmonary congestion, conductance across the patient's lung increases, causing a corresponding decrease in impedance indicative of the level of thoracic fluid accumulation. Such implantable cardiac devices can also be interrogated by hospital clinicians, allowing the hospital clinicians to monitor the patient's fluid status and to receive early warnings of changes that may signal an impending fluid overload. Based on the patient's monitored fluid status, the hospital clinicians may then determine whether or not it would be appropriate to readmit the patient to the hospital for further monitoring and/or treatment.

Convention implantable cardiac devices have several disadvantages. For example, conventional implantable cardiac devices typically include one or more sensors configured to provide a single or limited number of sensing modalities, such as a modality for detecting a human subject's fluid retention. However, monitoring and/or tracking the CHF status of a human subject based on just a single or limited number of sensing modalities can often lead to false positives, resulting in unnecessary hospital readmissions that can increase healthcare costs. In addition, the implantable nature of such conventional cardiac devices can increase surgical risks, as well as the incidence of infection. Furthermore, the implantable nature of such conventional cardiac devices limits the availability of the devices to patients, since only patients qualified for the surgery to insert the implant can receive the device.

To overcome these disadvantages, external and non-invasive devices for at-home detection and monitoring of CHF and/or other health conditions, such as chronic obstructive pulmonary disease (COPD), are being developed. External devices can provide multiple sensing modalities that non-invasively gather data, and can at least partially analyze, trend, and/or reduce data from the various sensing modes. For example, an external device may include one or more electrodes, heart sounds sensors, ultrasound sensors, and photoplethysmography sensors. A multi-modality sensing device increases the positive detection of potentially problematic CHF conditions while decreasing false positives, which can reduce the number of unnecessary hospital readmissions, shorten hospital stays, and reduce hospital costs. Moreover, an external device that can be conveniently employed by a patient following discharge from the hospital allows the patient as well as hospital clinicians to monitor the subject's CHF and/or COPD status without the risks or surgery and infection.

### BRIEF DESCRIPTION OF THE DRAWINGS

FIG. 1 illustrates a sensor device positioned on a chest of a wearer, according to some embodiments of the present disclosure;

FIG. 2 illustrates a wearer of the sensor device in a first body posture, according to some embodiments of the present disclosure;

FIG. 3 illustrates a wearer of the sensor device in a second body posture, according to some embodiments of the present disclosure;

FIG. 4 is a block diagram illustrating components of a sensor system with a user interface, according to some embodiments of the present disclosure;

FIG. 5 is an example illustration of a user interface housing of the sensor device with audio and visual outputs, according to some embodiments of the present disclosure;

FIG. 6 is a flowchart showing a method of guiding a user through a sequence of measurements with the sensor device, according to some embodiments of the present disclosure.

### DESCRIPTION OF EXAMPLE EMBODIMENTS OF THE DISCLOSURE

Overview

The systems, methods and devices of this disclosure each have several innovative aspects, no single one of which is solely responsible for all of the desirable attributes disclosed herein. Details of one or more implementations of the subject matter described in this specification are set forth in the description below and the accompanying drawings.

As described above, external devices for at-home monitoring of CHF, COPD, or other health conditions provide several advantages for patients and clinicians, including eliminating the need for surgical implementation which increases risks and costs to the patient, and collecting a broader set of data with multiple sensing modalities. For example, a doctor may prescribe a device to a patient and request that the patient use the device to obtain measurements twice per day, e.g., at the morning and at night. A patient using an external, removable device at home must be able to properly adhere the device to their chest and position their body properly so that the device collects usable data.

For example, an external device may include two or more electrodes for measuring thoracic impedance across the wearer's chest and an acoustic sensor to measure the wearer's heart sound (e.g., S3 or S4 heart sounds). In some embodiments, e.g., for COPD detection, the acoustic sensor, or one or more additional acoustic sensors, may capture lung sounds. In some embodiments, the electrodes, or one or more additional electrodes, may be used to collect electrocardiogram (ECG) signals. If the electrodes, acoustic sensors, or other sensors are not in proper contact with the wearer's chest, the device may not capture the intended signals. For example, if the electrodes are improperly adhered, they may not accurately measure impedance across the portion or portions of the wearer's chest; as another example, acoustic devices that are not in full contact with the wearer's chest may capture attenuated heart sounds and/or lung sounds, or fail to capture the target sounds.

In some embodiments, the external device may be configured to capture measurements in a series of body postures. For example, the external device captures a first set of measurements while the user is seated upright, and a second set of measurements while the user's chest is at an angle, e.g., in Fowler's position. The user must independently move through these postures and stay in the correct postures for the duration of each set of measurements.

The audio and visual user interface described herein guides users through the process of adhering the external sensor to their chest, beginning the measurement sequence, moving through a series of body postures, and ending the measurement sequence. For example, the sensor system provides audio and visual alerts to the user if the sensor is not properly adhered, and if the user is not in an appropriate body posture for a particular set of measurements. The sensor system may provide an error resolution tone indicating that a user has properly resolved the contact or body posture issue. The sensor system may also provide various audio and/or visual outputs as the user moves through the measurement sequence, to guide the user through the process and ensure the user that the measurements are being taken. Such outputs may include a cue that the sensor system is currently taking measurements, a cue for the user to change body posture, and a cue that the sensor system is finished and the user can remove the external sensor. Another output can include a cue for the user to recharge the external sensor.

The cues are designed to be easily detectable and understood by a wide range of users. It is anticipated that some users of the external sensor device may have audio and/or visual impairments. To account for this, audio alerts are selected in a range of frequencies that is detectable to many or most users, including users with some degree of hearing loss. If the sensor system includes an acoustic sensor (e.g., for capturing heart sounds), the range of frequencies for the audio sounds may also be selected so as to not overlap with the frequencies detected by the acoustic sensor. The audio alerts are also selected so that their meaning can be intuitively understood by users, e.g., a series of tones of increasing pitch instruct a user to lean forward, and a series of tones of decreasing pitch instruct a user to lean backward.

The external device may also provide corresponding visual cues, which may provide redundant signals for users with a greater degree of hearing loss who cannot hear the tones. Each alert or cue may have a corresponding color or color sequence. The colors selected for the visual alerts are colors that are distinguishable to many or most users, including users with color deficiency. For example, yellow, orange, pink, and green are distinguishable from each other to most users with different forms of color deficiency. Furthermore, the colors may be selected so that their meaning can be easily understood by users, e.g., a green light or green flash indicates that an issue (e.g., a bad electrode contact or incorrect body posture) has been resolved, or a measurement has started, while orange and yellow lights or flashes are used to represent different issues, e.g., body posture or contact issues.

Example Sensor Device

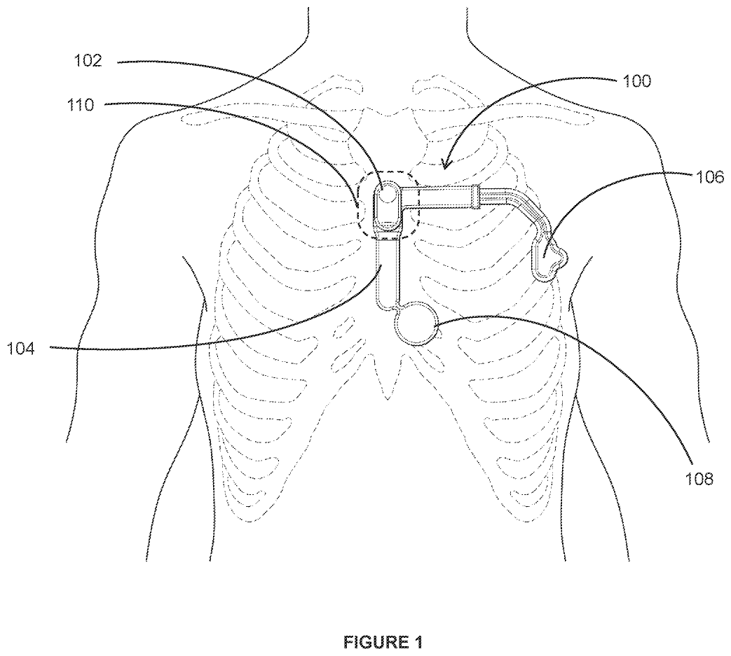

FIG. 1 illustrates a sensor device 100 positioned on a chest of a wearer, according to some embodiments of the present disclosure. The sensor device 100 includes a user interface (UI) housing 102 that is physically connected and electrically coupled to two islands 106 and 108. A base material 104, such as a rubber or other flexible material, connects the UI housing 102 to the two islands 106. One of the islands 106 is positioned under the left arm area, near the midaxillary line. The other island 108 is positioned just to the left of the sternum, e.g., at the left sternal border. The UI housing 102 and the islands 106 and 108 have an adhesive backing for adhering the sensor device 100 to the user's chest during a measurement procedure. A patient may wear the sensor device 100 during measurement sessions, e.g., for a period of several minutes, one or more times a day, and remove the sensor device 100 after each measurement session. The adhesive backing may be replaceable, e.g., once per week.

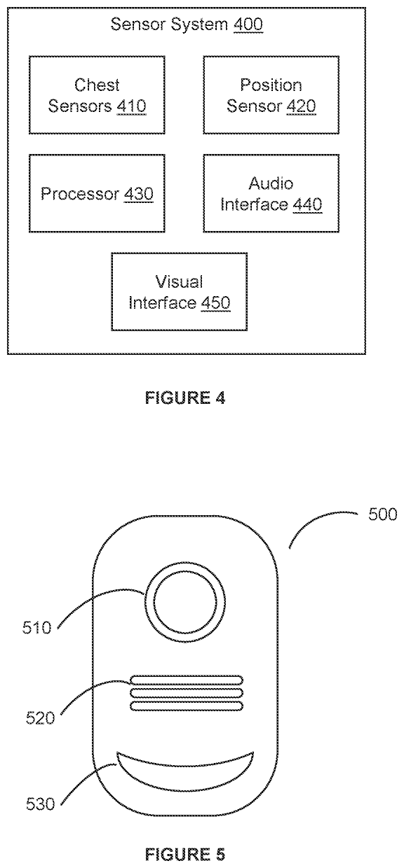

The sensor device 100 includes various sensors to capture data that may assist with management of one or more cardiopulmonary conditions, such as CHF or COPD. In one embodiment, the UI housing 102, which is positioned near the apex of the patient's heart, includes an electronic stethoscope or other acoustic sensor for capturing heart sounds. The islands 106 and 108 each include one or more electrodes, e.g., for capturing thoracic impedance measurements, ECG measurements, or other electrical measurements. In some embodiments, the sensor device 100 (e.g., the UI housing 102) includes additional sensors, such as a temperature sensor to measure body temperature, and a position sensor to measure tilt (body posture). The UI housing 102 further includes control circuitry for controlling the sensor device 100, e.g., to control the electrodes and other sensors, and to capture and store data. The UI housing 102 further includes one or more user interface features, such as a button for the patient to indicate when to start collecting measurements, and light, sound, or other outputs for providing information or alerts to the patient. The user interfaces are described in greater detail with respect to FIGS. 4-6. The UI housing 102 may include a battery and one or more electronic interfaces, e.g., to connect to a base station to export measurement data, receive firmware updates, receive power, etc.

The sensors in the sensor device 100 capture a variety of data that can be used by a doctor to identify potential cardiopulmonary issues or other health concerns. The sensor device 100 or a connecting device, such as a base station, may transmit measurements from the sensor device 100 to a data portal (e.g., a web portal) available to the patient's doctor. This allows the doctor to remotely review the patient's measurements on a regular basis and, for example, determine whether to admit the patient to the hospital for further monitoring, or to change the patient's treatment plan. The sensor device 100 may capture, for example, skin temperature, thoracic impedance, change in thoracic impedance, respiration data, and tidal volume. The sensor device 100 (e.g., using the electrodes in the islands 106 and 108) also may capture an ECG and derive various metrics from the ECG, such as heart rate, and other physiological parameters. In some embodiments, some of these metrics may be derived by another device that receives data from the sensor device 100, e.g., computation may be done by a base station that connects to the sensor device 100.

To accurately capture measurements indicative of the patient's cardiopulmonary health, the sensor device 100 must be properly positioned on and adhered to the patient's chest. For example, if the UI housing 102 includes an electronic stethoscope, it can most accurately capture the heart sounds when positioned within a given range of the apex of the patient's heart. An example acceptable range 110 for the placement of the UI housing 102 is shown in FIG. 1; in FIG. 1, the UI housing 102 is placed in the center of this range, but the “correct” placement may include the range 110, e.g., of a centimeter or a few centimeters in the horizontal and/or vertical directions around the position of the UI housing 102 in FIG. 1. In other embodiments, the range may be smaller or greater, or the size of the range may differ between the horizontal and vertical directions (e.g., the range of acceptable positions may be greater in the vertical direction than in the horizontal direction). Proper positioning of the UI housing 102 can assist a patient in properly placing the islands 106 and 108. As noted above, the electrodes in the islands 106 and 108 are positioned on or near the midaxillary line and the left sternal border, respectively (e.g., within a few centimeters of these lines), to obtain a strong ECG signals and/or other measurements. The electrodes may also have target vertical positions relative to the patient's ribs, for example, the island 106 may be placed near the fifth intercostal space, and the island 108 may be placed near the fourth intercostal space.

Example Body Postures for Cardiopulmonary Measurements

In some implementations, the sensor device 100 obtains measurements from a wearer while the wearer is in multiple different body postures. The different body postures may include different angles of the wearer's torso relative to a horizontal plane, e.g., the ground or a bed. A patient may be instructed (e.g., by the patient's doctor) to have the sensor device 100 take a set of measurements in a first posture, move to a second posture, and have the sensor device 100 take a set of measurements in the second posture. The user interface of the sensor device 100 may help guide the patient through multiple postures, e.g., by instructing a wearer when to change postures, and providing signals to the wearer to lean forward or backward if the wearer is not positioned correctly.

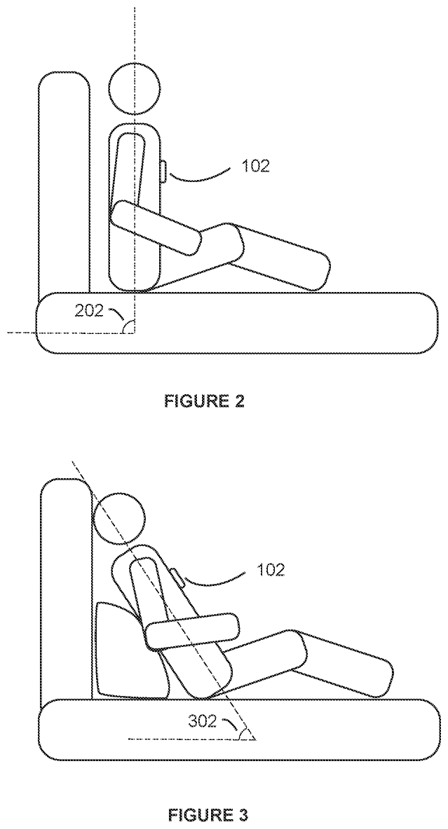

FIGS. 2 and 3 illustrate a wearer of the sensor device 100 in two different example body postures. FIG. 2 shows a first example body posture, in which the wearer's torso vertical or upright. An angle 202 between the wearer's torso and a bed that the wearer is resting on is around 90°. This position is referred to as a high Fowler's position. More generally, a high Fowler's position may include torso angles in a range between 60° and 90°. FIG. 3 shows a second example body posture, in which the wearer is in a standard Fowler position. In this example, an angle 302 between the wearer's torso and the bed is around 60°. Standard Fowler's position typically includes torso angles in a range between 45° and 60°.

In some embodiments, the sensor device 100 collects a first set of measurements while the wearer is seated upright, as shown in FIG. 2, and a second set of measurements while the wearer is in a standard Fowler's position, as shown in FIG. 3. In alternate embodiments, the sensor device 100 may collect measurements in one or more additional or alternate body postures, such as a semi-Fowler position (e.g., a torso angle between 30° and 45°, or between 15° and 45°), a low Fowler position (e.g., a torso position between 0° and 15°), or a fully reclining position (e.g., around 0°).

Example Sensor System

FIG. 4 is a block diagram illustrating components of a sensor system 400 with a user interface, according to some embodiments of the present disclosure. The sensor system 400 may be or include the sensor device 100, or the sensor system 400 may be or include a different chest-worn sensor for obtaining chest measurements of a wearer. The sensor system 400 includes chest sensors 410, a position sensor 420, a processor 430, an audio interface 440, and a visual interface 450. In alternative configurations, different, fewer, and/or additional components may be included in the sensor system 400 from those shown in FIG. 4. Furthermore, the functionality described in conjunction with one or more of the components shown in FIG. 4 may be distributed among the components in a different manner than described. For example, the sensor system 400 may be a single device (e.g., the sensor device 100), and certain functions of the processor 430 may be performed by an external processor, e.g., a processor in a base station that connects to the sensor system 400. FIG. 5, discussed below, illustrates an example device housing that may include some or all of the components of the sensor system 400.

The chest sensors 410 capture data that may assist with management of one or more cardiopulmonary conditions, such as CHF or COPD. For example, as described with respect to FIG. 1, the chest sensors 410 may include an electronic stethoscope or other acoustic sensor for capturing heart sounds, and one or more electrodes for capturing thoracic impedance measurements, ECG measurements, or other electrical measurements. The chest sensors 410 may further include any other types of sensors described above, e.g., with respect to FIG. 1. At least one of the chest sensors 410 (e.g., an acoustic sensor and/or one or more electrodes) may be located within a UI housing, e.g., the UI housing 102, or the UI housing shown in FIG. 5.

The position sensor 420 measures a tilt or orientation of the sensor system 400, which corresponds to a body posture of the wearer. The position sensor 420 may be located proximate to one or more of the chest sensors 410, e.g., within the UI housing 102, or the UI housing shown in FIG. 5. The position sensor 420 may include an accelerometer, a gyroscope, and/or another device for determining the orientation of the position sensor 420 relative to a ground or relative to the direction of gravity. If the portion of the sensor system 400 housing the position sensor 420 (e.g., the UI housing 102) is oriented parallel to the wearer's torso (e.g., the UI housing 102 is oriented vertically when the wearer is in an upright position), the position sensor 420 may measure the angle of the wearer's torso relative to a horizontal plane, e.g., the angle 202 or 302 shown in FIGS. 2 and 3. In some cases, based on the wearer's body shape, the UI housing holding the position sensor 420 may be angled relative to the direction of the wearer's torso (e.g., if the wearer's belly prevents the UI housing 102 from being oriented parallel to the torso). In this case, the wearer's doctor or other medical personnel may adjust one or more settings for the position sensor 420 so that the position sensor 420 can accurately measure the orientation of the wearer's torso, taking the wearer's body shape into account.

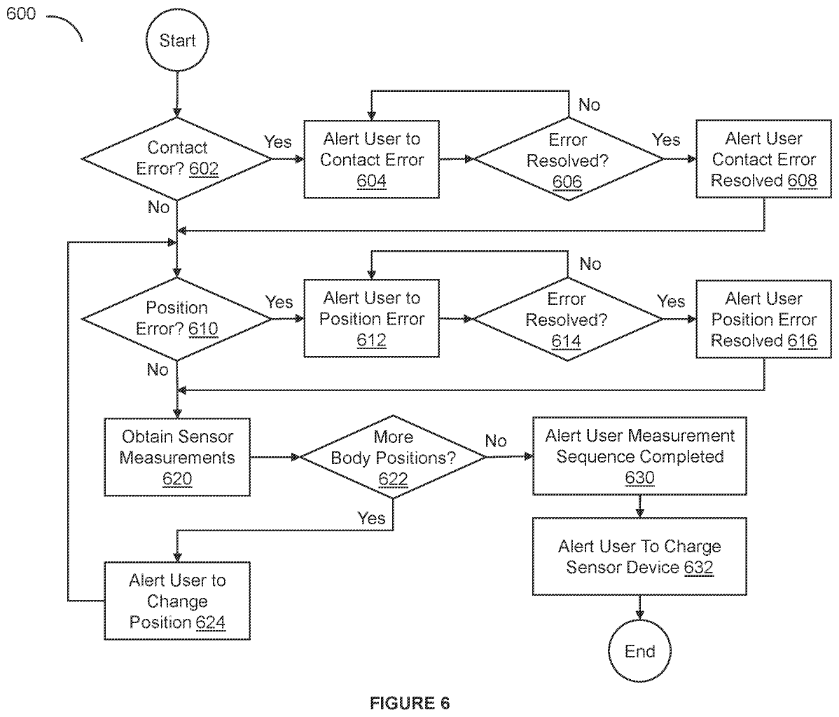

The processor 430 controls the other components of the sensor system 400, including the chest sensors 410, the position sensor 420, the audio interface 440, and the visual interface 450. The processor 430 may instruct chest sensors 410 to obtain sensor measurements. The processor 430 may perform processes for guiding the wearer through a measurement sequence. For example, the processor 430 instructs the position sensor 420 to take a position measurement, compares the position measurement to an expected body posture, and instructs the audio interface 440 and/or visual interface 450 to provide cues to the wearer to change their body posture if the measured position does not match an expected body posture for a particular set of measurements. Other functionalities of the processor 430 are described in relation to FIG. 6.

The audio interface 440 provides audio cues to a user to assist the user in taking a set or sequence of measurements. The audio interface 440 may include a speaker configured to output tones across a range of frequencies. The frequencies may be selected so that they are detectable to many or most users, including users with some degree of hearing loss. Because high-pitch hearing loss (i.e., loss of the ability to hear tones at higher frequencies) is the most common type of hearing loss, the frequencies may be selected to include lower pitch tones, e.g., tones below 2000 Hz, or tones below 1000 Hz. Furthermore, if the chest sensors 410 includes an acoustic sensor, the range of frequencies for the audio interface 440 may be selected to not overlap with the frequencies detected by the acoustic sensor. For example, if the acoustic sensor detects frequencies at around 500 Hz or lower (e.g., 512 Hz and lower), the frequencies output by the audio interface 440 include tones with pitches above the acoustic sensor range (e.g., above 512 Hz). If the audio cues, or certain audio cues, are only output at times when the acoustic sensor is not measuring heart sounds, the frequency range of the audio interface 440 may overlap with the range of frequencies measured by the acoustic sensor. Particular examples of audio outputs used for various cues are described with respect to FIG. 6.

The visual interface 450 provides visual cues to a user to assist the user in taking a set or sequence of measurements. The visual interface 450 may provide redundant cues for users with a greater degree of hearing loss who cannot hear the tones. The visual interface 450 may include one or more LEDs, e.g., an LED that can emit multiple different colors, or multiple LEDs, each configured to emit a single color. The different colors can be used independently or in combination to provide different visual cues. For example, each cue may have a corresponding color or sequence of colors. Further, each cue may have a corresponding pattern, e.g., two or three flashes of a single color, or a flash of one color followed by a flash of a different color. The visual interface 450 may emit colors that are distinguishable to many or most users, including users with color deficiency. For example, yellow, orange, pink, and green are distinguishable from each other to most users with different forms of color deficiency. Particular examples of visual outputs used for various cues are described with respect to FIG. 6.

Example UI Housing with Audio and Visual Interface

FIG. 5 is an example illustration of a UI housing of the sensor system 400 with audio and visual outputs, according to some embodiments of the present disclosure. The UI housing 500 may be an example of the UI housing 102 shown in FIG. 1. The UI housing 500 includes a button 510, a speaker 520, and a light 530. The button 510 may enable user input to the sensor system 400, and the user input (e.g., a signal indicating that the button 510 was pressed) may be provided to the processor 430. For example, the user may press the button 510 to signal to the sensor system 400 that the user is ready for a measurement sequence to begin. As another example, after the sensor system 400 has completed one set of measurements and the user has moved from one body posture to another, the user may press the button 510 to signal to the sensor system 400 that the user is in position and ready for the next set of measurements to begin.

The speaker 520 is an example of the audio interface 440. The speaker 520 outputs various audio tones to provide audio cues to the user, as described with respect to FIG. 4 and described further below with respect to FIG. 6. The light 530 is an example of the visual interface 450. The light 530 outputs various visual cues (e.g., different colored lights) to the user, as described with respect to FIG. 4 and described further below with respect to FIG. 6.

Examples Process Performed by Sensor Device

FIG. 6 is a flowchart showing a method 600 of guiding a user through a sequence of measurements with the sensor device, according to some embodiments of the present disclosure. The method 600 may be performed by the sensor system 400. The method 600 may begin after the user has adhered the sensor system 400 to the user's chest and pressed the button 510 to begin the measurement sequence.

The sensor system 400 (e.g., the processor 430) determines 602 whether there is a contact issue between one or more of the chest sensors 410 and the user. For example, the chest sensors 410 takes a sample measurement to determine whether there is a proper connection between the chest sensor 410 and the user's chest. For example, if the acoustic sensor detects no heart sound, or the heart sound is weaker than expected, this may indicate that the UI housing 102 is not firmly adhered to the user's chest, or the UI housing 102 may not be positioned correctly. As another example, if an electrode does not detect an electrical signal, or the electrical signal is weaker than expected, this may indicate that the electrode is not firmly adhered to the user's chest, or the electrode is not positioned correctly.

If the processor 430 detects a contact issue, the processor 430 instructs the audio interface 440 and/or the visual interface 450 to alert 604 the user to the contact issue. For example, the processor 430 instructs the audio interface 440 to output a set of tones used to represent “contact issue,” e.g., a set of four tones of the same length and same pitch. Four tones may be selected because “contact issue” has four syllables, and a repeating pitch may be naturally interpreted to a user as an error signal. In one embodiment, the tones have the pitch G5 (about 784 Hz). In other embodiments, different patterns or pitches may be used. In addition or alternatively, the processor 430 instructs the visual interface 450 to output a light or light sequence that represents “contact issue,” e.g., an orange light, or a set of orange flashes (e.g., four flashes output simultaneously with the four tones output by the audio interface 440). In response to the contact issue, the user may press down on one or more portions of the sensor system 400, e.g., the UI housing 102 and the electrode islands 106 and 108. If this does not resolve the issue, the user may need to take further action, such as checking that the adhesives are applied properly to the device, cleaning their skin under the adhesives, or trimming their chest hair.

After the audio interface 440 and or visual interface 450 outputs the alert, the processor 430 determines 606 whether the contact issue has been resolved. The processor 430 may use a similar process to determining 602 whether there is a contact issue as described above. The processor 430 may wait for a set amount of time (e.g., 10 or 20 seconds) between alerting 604 the user to the contact issue and determining 606 whether the issue is resolved to give the wearer an opportunity to resolve the contact issue. If the processor 430 determines that the contact issue has been resolved, the processor 430 instructs the audio interface 440 and/or the visual interface 450 to alert 608 the user that the contact issue has been resolved. For example, the processor 430 instructs the audio interface 440 to output a set of tones used to represent “OK,” e.g., a set of two tones of the same length and same pitch. Two tones may be selected because “OK” has two syllables. The pitch may be lower than the issue notification pitch, with a lower pitch being interpreted as more of a reassuring cue rather than an alert. In one embodiment, the tones have the pitch B4 (about 494 Hz). In other embodiments, different patterns or pitches may be used. In addition or alternatively, the processor 430 instructs the visual interface 450 to output a light or light sequence that represents “OK,” e.g., a green light, or a set of green flashes (e.g., two green flashes output simultaneously with the two tones output by the audio interface 440).

After alerting the user that the contact issue is resolved, or if no contact issue was detected, the process 600 proceeds to determine 610 whether there is a posture issue, e.g., whether the user is in the correct posture or within a range of correct postures for a particular set of measurements. A user may be instructed to be in a particular body posture during the chest measurements. As noted above, the sensor system 400 may be used to obtain a sequence of measurements, with two or more sets of measurements taken while the user is in different body postures. The processor 430 receives data from the position sensor 420 and uses this data determine a body angle of the user. The processor 430 compares the body angle of the user to an expected body angle, or to a range of expected body angles (e.g., between 80° and 95°, or between 45° and 60°). The processor 430 determines whether the body angle matches an expected body angle (e.g., the body angle is within ±5° of an expected body angle) or is within the range of expected body angles.

If the user's body angle does not match an expected body angle or range of expected range of body angles, the processor 430 instructs the audio interface 440 and/or the visual interface 450 to alert 612 the user to the body posture issue. For example, the processor 430 instructs the visual interface 450 to output a light or light sequence that represents “posture issue,” e.g., a yellow light, or a set of yellow flashes (e.g., three yellow flashes output simultaneously with three tones output by the audio interface 440, as described below).

In some embodiments, the processor 430 may determine if the body angle is larger than an expected body angle or larger than the high end of the body angle range, and the user should lean backward; or if the body angle is smaller than an expected body angle or smaller than the low end of the body angle range, and the user should lean forward. The processor 430 may instruct the audio interface 440 to provide different audio cues based on whether the user should lean forward or lean backward. For example, if the processor 430 determines that the user should lean forward, the processor 430 may instruct the audio interface 440 to output a set of tones used to represent “lean forward,” e.g., a set of three tones of the increasing pitch. For example, the pitches (in order) may be C5, E5, and G5. If the processor 430 determines that the user should lean backward, the processor 430 may instruct the audio interface 440 to output a set of tones used to represent “lean backward,” e.g., a set of three tones of the decreasing pitch. For example, the pitches (in order) may be G4, E4, and C4. As noted above, the audio cues are selected to be easily learned or naturally interpreted. In other embodiments, different patterns or pitches may be used.

After the audio interface 440 and/or visual interface 450 outputs the alert, the processor 430 determines 614 whether the posture issue has been resolved. The processor 430 may use a similar process to the process of determining 610 whether there is a posture issue as described above. The processor 430 may wait for a set amount of time (e.g., 5 or 10 seconds) between notifying 612 the user to the posture issue and determining 614 whether the issue is resolved to give the user an opportunity to respond to the notification and change postures. If the processor 430 determines that the posture issue has been resolved, the processor 430 instructs the audio interface 440 and/or the visual interface 450 to notify 616 the user that the posture issue has been resolved. For example, as described with respect to the notification 608, the processor 430 instructs the audio interface 440 to output a set of tones used to represent “OK,” e.g., a set of two tones of the same length and same pitch, e.g., the pitch B4 (about 494 Hz). In addition or alternatively, the processor 430 instructs the visual interface 450 to output a light or light sequence that represents “OK,” e.g., a green light, or a set of green flashes (e.g., two green flashes output simultaneously with the two tones output by the audio interface 440). In other embodiments, different audio patterns, pitches, or light outputs may be used; in some cases, the notification 616 indicating that an incorrect body posture (tilt angle) issue is resolved is different from the contact issue notification 608 indicating that the contact issue is resolved.

While the contact issue sequence (602-608) is shown in FIG. 6 as being performed prior to the posture issue sequence (610-616), in other embodiments, these two sequences may be switched, i.e., the processor 430 may first check for a posture issue, and then check for a contact issue.

After notifying the user that the incorrect body posture notification is resolved, or if no body posture issue was detected, the processor 430 instructs the chest sensors 410 to obtain 620 sensor measurements, e.g., a set of acoustic measurements and a set of impedance measurements. In some embodiments, the processor 430 instructs the audio interface 440 and/or visual interface 450 to output a cue indicating that measurements are being taken. For example, the processor 430 instructs the audio interface to output a set of tones used to represent “start measurement,” e.g., a set of four tones of increasing pitch. For example, the pitches (in order) may be C5, E5, G5, C6, which provides a positive, reassuring sequence to let the user know that the measurement is starting correctly. This may be accompanied by a visual cue, e.g., one or more green flashes. As another example, a “beat” cue (e.g., a single tone and/or single green light flash) may be output periodically (e.g., every 5 or 10 seconds) during a set of measurements to indicate that measurements are being obtained and recorded.

After a set of measurements has completed, the processor 430 may determine 622 whether any additional sets of measurements are to be taken in different body postures. If another set of measurements is to be taken (e.g., if the sensor device 400 has taken a first set of measurements while the user is upright, and the sensor system 400 should take a second set of measurements while the user is in Fowler's position), the processor 430 instructs the audio interface 440 and/or visual interface 450 to alert 624 the user to change their posture. For example, the processor 430 instructs the audio interface 440 to output a set of tones used to represent “change position,” e.g., a set of four tones of varying pitch, e.g., two higher pitched tones followed by two lower pitched tones. Four tones may be selected because “change positions” has four syllables, and a varying pitch may be naturally interpreted to a user as a cue to take an action (here, change positions) rather than an error signal. In one embodiment, the tones have the pitches C6, C6, C4, C4. In other embodiments, different patterns or pitches may be used. In addition or alternatively, the processor 430 instructs the visual interface 450 to output a light or light sequence that represents “change position,” e.g., a pink light, or a set of pink flashes (e.g., four pink flashes output simultaneously with the four tones output by the audio interface 440).

In the process shown in FIG. 6, after the alert 624 is provided, the process returns to the decision of determining 610 whether there is a posture issue for the new posture. In an alternate embodiment, the process instead returns to the decision of determining 602 whether there is a contact issue after the user has changed posture. In some embodiments, each time the user is to change posture, the processor 430 waits for a signal indicating that the user has pressed the button 510 to proceed with the determination 610 or 602.

If the processor 430 determines 662 that no more sets of measurements are to be taken, the processor 430 instructs the audio interface 440 and/or visual interface 450 to alert 630 the user to that the measurement sequence has completed. For example, the processor 430 instructs the audio interface 440 to output a set of tones used to represent “end measurement,” e.g., a set of four tones of decreasing pitch. Tones of decreasing pitch may be naturally interpreted to a user as a cue that the measurement sequence is complete. In one embodiment, the tones have the pitches C6, G5, E5, C5. In other embodiments, different patterns or pitches may be used. In addition or alternatively, the processor 430 instructs the visual interface 450 to output a light or light sequence that represents “end measurement,” e.g., a green light, or a set of green flashes (e.g., four green flashes output simultaneously with the four tones output by the audio interface 440).

In some embodiments, after the user has removed the sensor device (e.g., detected based on one or more of the chest sensors 410 losing contact with the user), or some time after outputting the alert 630 that the measurement sequence is completed, the processor 430 instructs the audio interface 440 and/or visual interface 450 to alert 632 the user to charge the sensor device. For example, the processor 430 instructs the audio interface 440 to output a set of tones used to represent “plug it in,” e.g., a set of three tones of varying pitches. In one embodiment, the tones have the pitches C6, D4, C4. In other embodiments, different patterns or pitches may be used. In addition or alternatively, the processor 430 instructs the visual interface 450 to output a light or light sequence that represents “plug it in,” e.g., a combination of green and yellow light (e.g., alternating green and yellow flashes, or simultaneous green and yellow light). After this alert, the process 600 may end.

### Select Examples

Example 1 provides a sensor system including a chest sensor to be worn on a chest of a user; an audio interface to output a plurality of audio cues to the user, the audio cues relating to a measurement sequence performed by the sensor system; and a processor to instruct the chest sensor to capture measurement data while the use is in a first body posture; instruct the audio interface to output a first audio cue instructing the user to move to a second body posture; instruct the chest sensor to capture measurement data while the user is in the second body posture; and instruct the audio interface to output a second audio cue instructing the user that a measurement sequence is complete.

Example 2 provides the sensor system of Example 1, further including a positioning sensor to determine an orientation of the chest sensor, where the processor is further to determine, based on data from the positioning sensor, whether a body angle of the user matches an expected body angle for the first body posture; and in response to determining that the body angle does not match the expected body angle, instruct the audio interface to output a third audio cue instructing the user to adjust their body posture.

Example 3 provides the sensor system of Example 2, where the processor is to instruct the audio interface to output a first sound sequence in response to the body angle of the user being smaller than the expected body angle, and to instruct the audio interface to output a second sound sequence in response to the body angle of the user being greater than the expected body angle.

Example 4 provides the sensor system of Example 3, where the first sound sequence includes a set of tones of increasing frequency, and the second sound sequence includes a set of tones of decreasing frequency.

Example 5 provides the sensor system of Example 1, where the plurality of audio cues include tones in a range of 500 Hz and 4000 Hz.

Example 6 provides the sensor system of Example 1, where the chest sensor includes an acoustic sensor to capture a first range of frequencies, and plurality of audio cues include tones in a second range of frequencies that does not overlap with the first range of frequencies.

Example 7 provides the sensor system of Example 1, where the processor is further to determine, based on data from the chest sensor, whether there is a contact issue between the chest sensor and the user's chest; and in response to determining that there is a contact issue between the chest sensor and the user's chest, instructing the audio interface to output a third audio cue instructing the user to press on the chest sensor.

Example 8 provides the sensor system of Example 1, where the processor is further to detect an issue, where the issue is a posture issue or a contact issue; detect whether the issue has been resolved; and in response to determining that the issue has been resolved, instructing the audio interface to output a third audio cue indicating that the issue has been resolved.

Example 9 provides the sensor system of Example 1, further including a visual interface to output a plurality of visual cues, where one of the plurality of visual cues corresponds to one of the plurality of audio cues.

Example 10 provides the sensor system of Example 9, where each of the plurality of visual cues includes a light color and a light pattern.

Example 11 provides method involving retrieving sensor data from a chest-worn sensor device, the sensor device to obtain measurements related to health of a user; detecting, based on the sensor data, whether there is an issue in the sensor device; in response to detecting an issue, instructing an audio interface of the sensor device to output an audio cue alerting the user to the issue; detecting whether the issue has been resolved; and in response to detecting that the issue has been resolved, instructing the sensor device to capture measurement data.

Example 12 provides the method of Example 11, where the sensor data includes position data related to a body angle of the user, and detecting whether there is an issue in the sensor device includes determining, based on the position data, whether a body angle of the user matches an expected body angle.

Example 13 provides the method of Example 12, where instructing an audio interface of the sensor device to output an audio cue alerting the user to the issue includes instructing the audio interface to output a first sound sequence in response to the body angle of the user being smaller than the expected body angle; or instructing the audio interface to output a second sound sequence in response to the body angle of the user being greater than the expected body angle.

Example 14 provides the method of Example 11, where detecting whether there is an issue in the sensor device includes determining, based on the sensor data, whether there is a contact issue between the sensor device and the user's chest.

Example 15 provides the method of Example 11, further including in response to detecting that the issue has been resolved, instructing the audio interface to output a second audio cue indicating that the issue has been resolved.

Example 16 provides the method of Example 11, further including in response to detecting the issue, instructing a visual interface of the sensor device to output a visual cue alerting the user to the issue, the visual cue including at least one of a light color and a light pattern.

Example 17 provides a sensor system including a chest sensor to be worn on a chest of a user, the chest sensor to obtain measurements related to health of the user; a positioning sensor for determining an orientation of the chest sensor; a processor to determine a body angle of the user based on data from the positioning sensor and compare the body angle of the user to an expected body angle; and an audio interface to output a first audio cue in response to the body angle of the user being smaller than the expected body angle, and output a second audio cue in response to the body angle of the user being greater than the expected body angle.

Example 18 provides the sensor system of Example 17, further including a light interface to output a visual cue in response to the body angle of the user differing from the expected body angle.

Example 19 provides the sensor system of Example 17, where the first audio cue includes a set of tones of increasing frequency, and the second audio cue includes a set of tones of decreasing frequency.

Example 20 provides the sensor system of Example 17, the processor further to determine that the chest sensor has obtained a sequence of measurements in a first body posture, the first body posture including the expected body angle; and in response to determining that the chest sensor has obtained the sequence of measurements, instruct the audio interface to output a third audio cue, the third audio cue indicating to the user to move to a second body posture.

### OTHER IMPLEMENTATION NOTES, VARIATIONS, AND APPLICATIONS

It is to be understood that not necessarily all objects or advantages may be achieved in accordance with any particular embodiment described herein. Thus, for example, those skilled in the art will recognize that certain embodiments may be configured to operate in a manner that achieves or optimizes one advantage or group of advantages as taught herein without necessarily achieving other objects or advantages as may be taught or suggested herein.

In one example embodiment, any number of electrical circuits of the figures may be implemented on a board of an associated electronic device. The board can be a general circuit board that can hold various components of the internal electronic system of the electronic device and, further, provide connectors for other peripherals. More specifically, the board can provide the electrical connections by which the other components of the system can communicate electrically. Any suitable processors (inclusive of digital signal processors, microprocessors, supporting chipsets, etc.), computer-readable non-transitory memory elements, etc. can be suitably coupled to the board based on particular configuration needs, processing demands, computer designs, etc. Other components such as external storage, additional sensors, controllers for audio/video display, and peripheral devices may be attached to the board as plug-in cards, via cables, or integrated into the board itself. In various embodiments, the functionalities described herein may be implemented in emulation form as software or firmware running within one or more configurable (e.g., programmable) elements arranged in a structure that supports these functions. The software or firmware providing the emulation may be provided on non-transitory computer-readable storage medium comprising instructions to allow a processor to carry out those functionalities.

It is also imperative to note that all of the specifications, dimensions, and relationships outlined herein (e.g., the number of processors, logic operations, etc.) have only been offered for purposes of example and teaching only. Such information may be varied considerably without departing from the spirit of the present disclosure, or the scope of the appended claims. The specifications apply only to one non-limiting example and, accordingly, they should be construed as such. In the foregoing description, example embodiments have been described with reference to particular arrangements of components. Various modifications and changes may be made to such embodiments without departing from the scope of the appended claims. The description and drawings are, accordingly, to be regarded in an illustrative rather than in a restrictive sense.

Note that with the numerous examples provided herein, interaction may be described in terms of two, three, four, or more components. However, this has been done for purposes of clarity and example only. It should be appreciated that the system can be consolidated in any suitable manner. Along similar design alternatives, any of the illustrated components, modules, and elements of the FIGS. may be combined in various possible configurations, all of which are clearly within the broad scope of this Specification.

Note that in this Specification, references to various features (e.g., elements, structures, modules, components, steps, operations, characteristics, etc.) included in “one embodiment”, “example embodiment”, “an embodiment”, “another embodiment”, “some embodiments”, “various embodiments”, “other embodiments”, “alternative embodiment”, and the like are intended to mean that any such features are included in one or more embodiments of the present disclosure, but may or may not necessarily be combined in the same embodiments.

Numerous other changes, substitutions, variations, alterations, and modifications may be ascertained to one skilled in the art and it is intended that the present disclosure encompass all such changes, substitutions, variations, alterations, and modifications as falling within the scope of the appended claims. Note that all optional features of the systems and methods described above may also be implemented with respect to the methods or systems described herein and specifics in the examples may be used anywhere in one or more embodiments.

In order to assist the United States Patent and Trademark Office (USPTO) and, additionally, any readers of any patent issued on this application in interpreting the claims appended hereto, Applicant wishes to note that the Applicant: (a) does not intend any of the appended claims to invoke paragraph (f) of 35 U.S.C. Section 112 as it exists on the date of the filing hereof unless the words “means for” or “step for” are specifically used in the particular claims; and (b) does not intend, by any statement in the Specification, to limit this disclosure in any way that is not otherwise reflected in the appended claims.

What is claimed is:

1. A sensor system comprising:
a chest sensor to be worn on a chest of a user;
an audio interface to output a plurality of audio cues to the user, the audio cues relating to a measurement sequence performed by the sensor system; and
a processor to:
instruct the chest sensor to capture measurement data while the use is in a first body posture;
instruct the audio interface to output a first audio cue instructing the user to move to a second body posture;
instruct the chest sensor to capture measurement data while the user is in the second body posture; and
instruct the audio interface to output a second audio cue instructing the user that a measurement sequence is complete.

2. The sensor system of claim 1, further comprising a positioning sensor to determine an orientation of the chest sensor, wherein the processor is further to:
determine, based on data from the positioning sensor, whether a body angle of the user matches an expected body angle for the first body posture; and
in response to determining that the body angle does not match the expected body angle, instruct the audio interface to output a third audio cue instructing the user to adjust their body posture.

3. The sensor system of claim 2, wherein the processor is to instruct the audio interface to output a first sound sequence in response to the body angle of the user being smaller than the expected body angle, and to instruct the audio interface to output a second sound sequence in response to the body angle of the user being greater than the expected body angle.

4. The sensor system of claim 3, wherein the first sound sequence comprises a set of tones of increasing frequency, and the second sound sequence comprises a set of tones of decreasing frequency.

5. The sensor system of claim 1, wherein the plurality of audio cues comprise tones in a range of 500 Hz and 4000 Hz.

6. The sensor system of claim 1, wherein the chest sensor comprises an acoustic sensor to capture a first range of frequencies, and plurality of audio cues comprise tones in a second range of frequencies that does not overlap with the first range of frequencies.

7. The sensor system of claim 1, wherein the processor is further to:
determine, based on data from the chest sensor, whether there is a contact issue between the chest sensor and the user's chest; and
in response to determining that there is a contact issue between the chest sensor and the user's chest, instructing the audio interface to output a third audio cue instructing the user to press on the chest sensor.

8. The sensor system of claim 1, wherein the processor is further to:
detect an issue, wherein the issue is a posture issue or a contact issue;
detect whether the issue has been resolved; and
in response to determining that the issue has been resolved, instructing the audio interface to output a third audio cue indicating that the issue has been resolved.

9. The sensor system of claim 1, further comprising a visual interface to output a plurality of visual cues, wherein one of the plurality of visual cues corresponds to one of the plurality of audio cues.

10. The sensor system of claim 9, wherein each of the plurality of visual cues comprises a light color and a light pattern.

11. A method comprising:
retrieving sensor data from a chest-worn sensor device, the sensor device to obtain measurements related to health of a user;
detecting, based on the sensor data, whether there is an issue in the sensor device;
in response to detecting an issue, instructing an audio interface of the sensor device to output an audio cue alerting the user to the issue;
detecting whether the issue has been resolved; and
in response to detecting that the issue has been resolved, instructing the sensor device to capture measurement data.

12. The method of claim 11, wherein the sensor data comprises position data related to a body angle of the user, and detecting whether there is an issue in the sensor device comprises:
determining, based on the position data, whether a body angle of the user matches an expected body angle.

13. The method of claim 12, wherein instructing an audio interface of the sensor device to output an audio cue alerting the user to the issue comprises:
instructing the audio interface to output a first sound sequence in response to the body angle of the user being smaller than the expected body angle; or
instructing the audio interface to output a second sound sequence in response to the body angle of the user being greater than the expected body angle.

14. The method of claim 11, wherein detecting whether there is an issue in the sensor device comprises determining, based on the sensor data, whether there is a contact issue between the sensor device and a chest of the user.

15. The method of claim 11, further comprising:
in response to detecting that the issue has been resolved, instructing the audio interface to output a second audio cue indicating that the issue has been resolved.

16. The method of claim 11, further comprising:
in response to detecting the issue, instructing a visual interface of the sensor device to output a visual cue alerting the user to the issue, the visual cue comprising at least one of a light color and a light pattern.

17. A sensor system comprising:
a chest sensor to be worn on a chest of a user, the chest sensor to obtain measurements related to health of the user;
a positioning sensor for determining an orientation of the chest sensor;
a processor to:
determine a body angle of the user based on data from the positioning sensor; and
compare the body angle of the user to an expected body angle; and
an audio interface to output a first audio cue in response to the body angle of the user being smaller than the expected body angle, and output a second audio cue in response to the body angle of the user being greater than the expected body angle.

18. The sensor system of claim 17, further comprising a light interface to output a visual cue in response to the body angle of the user differing from the expected body angle.

19. The sensor system of claim 17, wherein the first audio cue comprises a set of tones of increasing frequency, and the second audio cue comprises a set of tones of decreasing frequency.

20. The sensor system of claim 17, the processor further to:
determine that the chest sensor has obtained a sequence of measurements in a first body posture, the first body posture comprising the expected body angle; and
in response to determining that the chest sensor has obtained the sequence of measurements, instruct the audio interface to output a third audio cue, the third audio cue indicating to the user to move to a second body posture.
